(19) United States
(12) Patent Application Publication
(10) Pub. No.: US 2010/0179526 A1
(43) Pub. Date: Jul. 15, 2010

(54) SYSTEMS AND METHODS OF MAKING AND USING A COILED COOLANT TRANSFER TUBE FOR A CATHETER OF A CRYOABLATION SYSTEM

(75) Inventor: David Lawrence, Saratoga, CA (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., Maple Grove, MN (US)

(21) Appl. No.: 12/685,794

(22) Filed: Jan. 12, 2010

Related U.S. Application Data
(60) Provisional application No. 61/144,066, filed on Jan. 12, 2009.

(51) Int. Cl. A61B 18/02

(52) U.S. Cl. 606/21

(57) ABSTRACT

A cryoablation catheter assembly includes an expansion element coupled to a distal portion of a catheter that is configured and arranged for insertion into patient vasculature. The catheter includes a guide tube, a coolant transfer tube, and at least one coolant outtake region that each extend along the catheter. The guide tube and the coolant transfer tube extend beyond the distal portion of the catheter. The coolant transfer tube includes a coiled distal that wraps around a portion of the guide tube and defines a plurality of spaced-apart spray apertures. The coolant transfer tube defines a lumen configured and arranged to receive and transfer coolant from a coolant source to the spray apertures. The expansion element is in fluid communication with the at least one coolant outtake region and the plurality of spray apertures.

### CROSS-REFERENCE TO RELATED APPLICATIONS

This application is a utility patent application based on a previously filed U.S. Provisional Patent Application Ser. No. 61/144,066 filed on Jan. 12, 2009, the benefit of which is hereby claimed under 35 U.S.C. §119(e) and incorporated herein by reference.

### TECHNICAL FIELD

The present invention is directed to the area of cryoablation systems and methods of making and using the systems. The present invention is also directed to cryoablation systems that include a catheter having a distally disposed expansion element, as well as systems and methods of using the catheters, expansion elements, and cryoablation systems.

### BACKGROUND

Cryoablation systems can be used to form cold-induced lesions on patient tissue. Cryoablation systems have proven therapeutic capabilities for a variety of diseases and disorders. For example, cryoablation systems have been used to treat a number of external benign and malignant skin conditions (e.g., warts, moles, skin cancers, and the like). Cryoablation systems have also been used to treat dysfunctional internal conditions (e.g., liver cancer, prostate cancer, stomach cancer, uterine cancer, cervical disorders, cardiac disorders, gastric reflux disease, gastro esophageal reflux disease, esophageal disease, and the like). Additionally, cryoablation systems have been used to reduce, or even eliminate, undesired electrical activity between adjacent cardiac tissues of the heart (arrhythmias).

One common type of arrhythmia, atrial fibrillation, is a result of abnormal electrical signals interfering with the normal electrical signal propagation along the tissues of the heart. Atrial fibrillation often originates near the ostia of the pulmonary veins. Cryoablation systems can be used to form lesions on patient tissue in proximity to the ostia, where the pulmonary veins open into the left atrium of the heart. The cold-induced lesions can effectively block the initiation or propagation of the abnormal electrical signals, thereby preventing the abnormal electrical signals from interfering with the normal electrical signal propagation along the tissues of the heart.

A cryoablation system can include a catheter configured and arranged for transporting coolant to and from a target location within a patient, an expansion element disposed at a distal portion of the catheter for ablating contacted patient tissue, a coolant source coupled to the catheter for supplying the coolant, and a control module for controlling or monitoring one or more of the operations of the system (e.g., controlling coolant flow, monitoring catheter pressure or temperature, or the like). The expansion element can be positioned at a target location in patient vasculature (e.g., the left atrium of the heart) and the coolant can be input to the catheter and directed to the expansion element. When the coolant contacts the expansion element, the coolant absorbs heat and expands, thereby causing the expansion element to expand and reduce in temperature to a level low enough to ablate patient tissue upon contact. The coolant flows out of the expansion element and back to a proximal end of the catheter. As the coolant flows out of the expansion element, the expansion element deflates and the catheter may be removed from the patient vasculature.

### BRIEF SUMMARY

In one embodiment, a cryoablation catheter assembly includes a catheter and an expansion element coupled to the distal portion of the catheter. The catheter has a distal portion, a proximal portion, and a longitudinal length. The catheter is configured and arranged for insertion into patient vasculature. The catheter includes a guide tube, a coolant transfer tube, and at least one coolant outtake region that each extend along the catheter. The guide tube extends beyond the distal portion of the catheter. The coolant transfer tube includes a coiled distal end that extends beyond the distal portion of the catheter. The coolant transfer tube defines a lumen configured and arranged to receive and transfer coolant from a coolant source. The coiled distal end defines a plurality of spaced-apart spray apertures. The coiled distal end is wrapped around a portion of the guide tube. The expansion element is coupled to the distal portion of the catheter and is in fluid communication with the at least one coolant outtake region. At least a portion of the coiled distal end of the coolant transfer tube extends into the expansion element such that the expansion element is in fluid communication with the plurality of spray apertures.

In another embodiment, a cryoablation system includes a catheter, an expansion element coupled to the distal portion of the catheter, a coolant source, a fluid-drawing source, and a control module. The catheter has a distal portion, a proximal portion, and a longitudinal length. The catheter is configured and arranged for insertion into patient vasculature. The catheter includes a guide tube, a coolant transfer tube, and at least one coolant outtake region that each extend along the catheter. The guide tube extends beyond the distal portion of the catheter. The coolant transfer tube includes a coiled distal end that extends beyond the distal portion of the catheter. The coolant transfer tube defines a lumen configured and arranged to receive and transfer coolant from a coolant source. The coiled distal end defines a plurality of spaced-apart spray apertures. The coiled distal end is wrapped around a portion of the guide tube. The expansion element is coupled to the distal portion of the catheter and is in fluid communication with the at least one coolant outtake region. At least a portion of the coiled distal end of the coolant transfer tube extends into the expansion element such that the expansion element is in fluid communication with the plurality of spray apertures. The coolant source is coupled to the coolant transfer tube. The fluid-drawing source is coupled to the at least one coolant outtake region. The control module is coupled to the catheter, the coolant source, and the fluid-drawing source. The control module includes a coolant flow controller that is configured and arranged for controlling the flow of coolant along the coolant transfer tube and the at least one coolant outtake region.

In yet another embodiment, a method for cryoablating patient tissue includes inserting a catheter into patient vasculature. The catheter has a distal portion, a proximal portion, and a longitudinal length. The catheter includes a guide tube extending along the catheter, a coolant transfer tube extending along the catheter, and at least one coolant outtake region. The guide tube and the coolant transfer tube extend beyond the distal portion of the catheter. The coolant transfer tube includes a coiled distal end that wraps around a portion of the guide tube. The catheter is guided in proximity to patient tissue to be ablated. Coolant is drawn from a coolant source such that coolant flows along the coolant transfer tube and is sprayed from spaced-apart spray apertures defined in the coiled distal end of the coolant transfer tube against an expansion element that is disposed at the distal portion of the catheter and that surrounds the coiled distal portion of the coolant transfer tube, thereby expanding the expansion element and reducing the temperature of the expansion element to a temperature sufficiently low enough to ablate patient tissue upon contact. Patient tissue is contacted with the expanded expansion element for a time period adequate to ablate tissue contacting the expansion element.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic partial cross-sectional and partial block diagram view of one embodiment of a cryoablation system, according to the invention;

FIG. 2A is a schematic longitudinal cross-sectional view of one embodiment of an expansion element coupled to a distal portion of a catheter of the cryoablation system of FIG. 1, the expansion element in a deflated configuration, according to the invention;

FIG. 2B is a schematic longitudinal cross-sectional view of one embodiment of an expansion element coupled to a distal portion of a catheter of the cryoablation system of FIG. 1, the expansion element an inflated configuration, according to the invention;

FIG. 3 is a schematic side view of one embodiment of a coolant transfer tube configured and arranged for insertion into the catheter of FIG. 2A, the coolant transfer tube having a coiled distal end that defines a plurality of spray apertures, according to the invention; and

FIG. 4 is a schematic longitudinal cross-sectional view of one embodiment of a distal portion of the catheter of FIG. 2A disposed in a sheath, according to the invention.

### DETAILED DESCRIPTION

The present invention is directed to the area of cryoablation systems and methods of making and using the systems. The present invention is also directed to cryoablation systems that include a catheter having a distally disposed expansion element, as well as systems and methods of using the catheters, expansion elements, and cryoablation systems.

Suitable cryoablation systems include, but are not limited to, an expansion element disposed on a distal end of a catheter configured and arranged for percutaneous insertion into a patient. Examples of cryoablation systems with catheters are found in, for example, U.S. Pat. Nos. 7,189,227; 7,101,368; 6,905,493; 6,755,822; and 6,666,858, all of which are incorporated by reference.

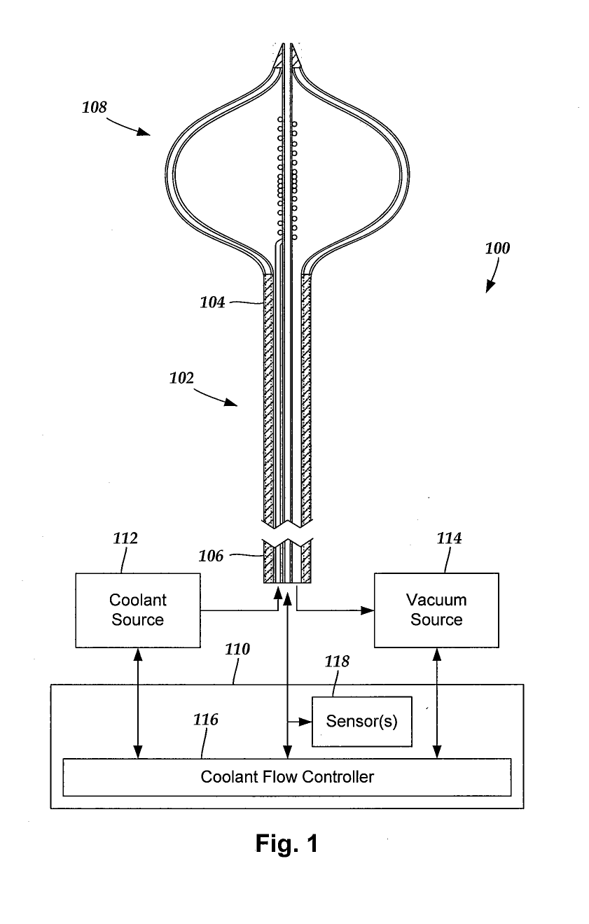

FIG. 1 illustrates schematically one embodiment of a cryoablation system 100. The cryoablation system 100 includes a catheter 102 with a distal portion 104 and a proximal portion 106. An expansion element 108 is coupled to the distal portion 104 of the catheter 102. A control module 110, a coolant source 112, and a fluid-drawing source 114 (e.g., a vacuum source, a pump, or the like) are each coupled to the proximal portion 106 of the catheter 102. The control module 110 includes a coolant flow controller 116 to control the flow of coolant within the catheter 102 to and from the expansion element 108. In at least some embodiments, the control module 104 also includes one or more sensors 118 for monitoring one or more conditions (e.g., pressure, temperature, or the like) within the catheter 102.

In at least some embodiments, the coolant source 112 includes a coolant under pressure. Many different coolants may be used to provide a low enough temperature to ablate tissue upon contact. In preferred embodiments, the coolant is a low freezing point liquid with a low vaporization temperature which may be input to the catheter 102 as a liquid that is sprayed into the expansion element 108, where the liquid coolant absorbs heat and is vaporized and atomized. Examples of suitable liquids include, for example, a liquefied gas (e.g., nitrogen, nitrous oxide, carbon dioxide, or the like), one or more chlorofluorocarbons, one or more hydrochlorofluorocarbons, ethanol mixtures, saline solutions, or the like. It will be understood that a combination of one or more coolants may also be used in the cryoablation system 100.

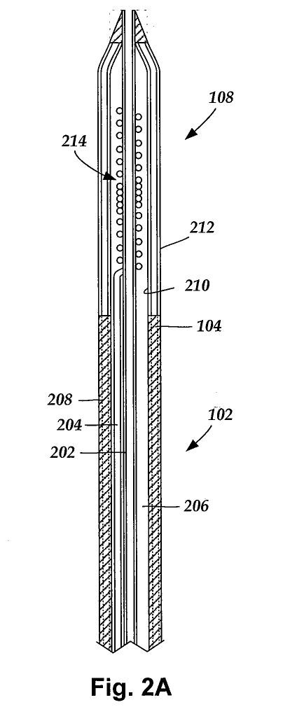

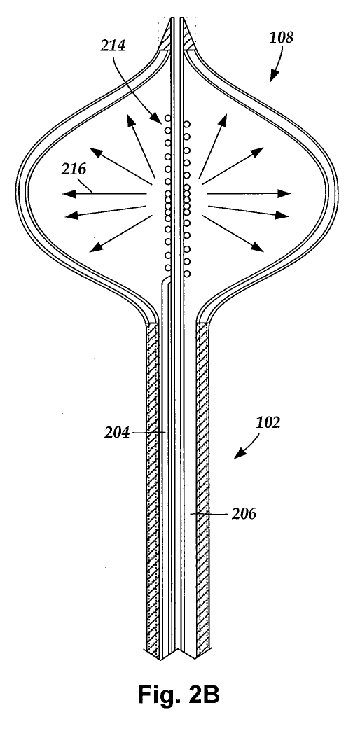

During a typical cryoablation procedure, the distal portion 104 of the catheter 102 is inserted into patient vasculature for delivery of the expansion element 108 to an ablation site. FIG. 2A is a schematic longitudinal cross-sectional view of one embodiment of the distal portion 104 of the catheter 102 and the expansion element 108. In FIG. 2A, the expansion element 210 is shown in a deflated configuration. The catheter 102 includes a guide tube 202, a coolant transfer lumen 204, and at least one coolant outtake region 206 each disposed in a flexible outer layer 208.

In some embodiments, the expansion element 108 includes a single member. In other embodiments, the expansion element 108 includes multiple members. For example, in at least some embodiments, the expansion element 108 includes an inner member 210 and an outer member 212 disposed over the inner member 210. FIGS. 1-2B and 4 show the expansion element 108 having two members. It will be understood that the expansion element 108 may, instead, only have a single member, or may have more than two members.

In at least some embodiments, a vacuum is maintained between the inner member 210 and the outer member 212. In at least some embodiments, a space between the inner member 210 and the outer member 212 is in fluid communication with the fluid-drawing source 114. In at least some embodiments, a proximal end of the expansion element 108 couples to the distal portion 104 of the catheter 104 such that a region within the inner member 210 is in fluid communication with the at least one coolant outtake region 206.

The expansion element 108 may be formed from any elastic or semi-elastic material, such as one or more thermoplastics (e.g., polyether block amide, or the like), or other plastics (e.g., nylon, urethane, or the like) that maintain elasticity over a wide range of temperatures, particularly at the temperature of the expanded coolant. In at least some embodiments, the expansion element 108 is semi-elastic, wherein the size of the expansion element 108 does not change in response to incremental changes in pressure that are below 5 psi (about 34.5×10³ Pa).

The guide tube 202 is configured and arranged to receive a stiffening member (e.g., a stylet, or the like) to facilitate guiding of the catheter 102 to a target location within patient vasculature by providing additional rigidity to the catheter 102. The guide tube 202 may be formed from any flexible material (e.g., a thermoplastic, or the like) that maintains elasticity over a wide range of temperatures, particularly at the temperature of the expanded coolant.

The guide tube 202 defines a lumen that extends along a longitudinal length of the catheter 102 from the proximal portion (106 in FIG. 1) of the catheter 102 to a position that is beyond the distal portion 104 of the catheter 102. In at least some embodiments, the guide tube 202 extends to a distal end of the expansion element 108 when the expansion element is in the deflated configuration. In at least some embodiments, the guide tube 202 extends to a distal end of the expansion element 108 when the expansion element is in an inflated configuration. In at least some embodiments, the distal end of the expansion element 108 is coupled to the guide tube 202.

The coolant outtake region 206 is configured and arranged to accommodate coolant exiting the expansion element 108. The coolant outtake region 206 extends along the longitudinal length of the catheter 102 from the proximal portion (106 in FIG. 1) of the catheter 102 to the expansion element 108. In some embodiments, the coolant outtake region 206 includes one or more tubes that define one or more lumens. In other embodiments, the coolant outtake region 206 includes one or more open regions within the outer layer 208 of the catheter 102 and exterior to the guide tube 202 and the coolant transfer tube 204. In at least some embodiments, a proximal end of the coolant outtake region 206 is in fluid communication with the fluid-drawing source (114 in FIG. 1).

The coolant transfer tube 204 extends along the longitudinal length of the catheter 102 from the proximal portion (106 in FIG. 1) of the catheter 102. The coolant transfer tube 204 defines a lumen. A proximal end of the lumen is coupled to the coolant source (112 in FIG. 1). The coolant transfer tube 204 includes a coiled distal end 214 that extends beyond the distal portion of the catheter 102. At least a portion of the coiled distal end 214 wraps around a portion of the guide tube 202. In at least some embodiments, the entire coiled distal end 214 is disposed distally from the distal portion 104 of the catheter 102. In at least some embodiments, a portion of the coiled distal end 214 is disposed in the distal portion 104 of the catheter 102.

In at least some embodiments, the coiled distal end 214 extends to a distal end of the expansion element 108 when the expansion element 108 is in a deflated configuration. In at least some embodiments, the coiled distal end 214 extends to the distal end of the expansion element 108 when the expansion element 108 is in an inflated configuration. In at least some embodiments, a distal end of the expansion element 108 is coupled to the coiled distal end 214. In at least some embodiments, the distal end of the guide tube 202 extends beyond the coiled distal end 214 of the coolant transfer tube 204.

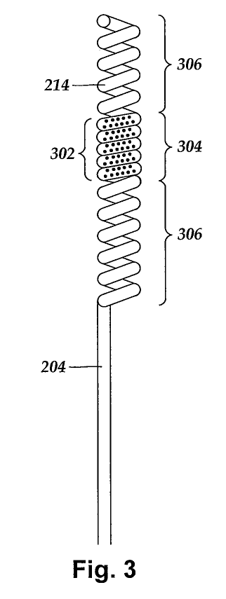

The coiled distal end 214 of the coolant transfer tube 204 defines a plurality of spaced-apart spray apertures (302 in FIG. 3) configured and arranged to output coolant from the coolant transfer tube 204 to the region inside the expansion element 108. In at least some embodiments, the coolant is output as a sprayed liquid that vaporizes and atomizes as the liquid is output from the coiled distal end 214 of the coolant transfer tube 204. In at least some embodiments, when the coolant enters the region within the expansion element 108, the expansion element 108 absorbs heat and expands, thereby reducing the temperature of the expansion element 108 to a temperature sufficiently low enough to ablate patient tissue upon contact.

The reduction in temperature of the expansion element 108 may be due to one or more of the Joule-Thompson effect or the latent heat of vaporization. The Joule-Thompson effect describes the cooling effect that comes about when a compressed non-ideal gas expands into a region of low pressure (e.g., within the expansion element 108). The latent heat of vaporization describes heat being absorbed as a result of the phase change from a liquid to a gas (e.g., the liquefied coolant vaporizing upon entering the expansion element 108).

FIG. 2B is a schematic longitudinal cross-sectional view of one embodiment of the expansion element 108 in an inflated configuration. Directional arrows, such as arrow 216, show the flow of coolant from the coiled distal end 214 of the coolant transfer tube 204 to an inner surface of the expansion element 108. The expanded gas dissipates down the catheter 102 along the coolant outtake region 206. In at least some embodiments, the fluid-drawing source (114 in FIG. 1) is used to draw the expanded, heated, and gaseous coolant along the coolant outtake region 206 from the expansion element 108 out the proximal end of the coolant outtake region 206.

FIG. 3 is a schematic side view of one embodiment of the coolant transfer tube 204. The coiled distal end 214 of the coolant transfer tube 204 defines a plurality of spaced-apart spray apertures 302. The sizes of the spray apertures 302 may all be similar or they may vary. The number of spray apertures 302 may vary, depending on any number of different reasons, (e.g., the specific type of ablation application, the type (or phase) of the coolant being used, or the like). In at least some embodiments, the spray apertures 302 are oriented such that the coolant output from the coolant transfer tube 204 is sprayed to one or more localized portions of the expansion element 108. In at least some embodiments, the spray apertures 302 are oriented such that the coolant output from the coolant transfer tube 204 is evenly sprayed along the expansion element 108.

When the distal portion 104 of the catheter 102 is inserted into the patient, the tortuous nature of patient vasculature may cause a number of lateral forces (i.e., forces not parallel to the longitudinal length of the distal portion 104 of the catheter 102) to push against the catheter 102, thereby causing the catheter 102 to bend, or even kink. Sometimes, one or more lateral forces are applied to the expansion element 108 such that the expansion element bends in relation to the distal portion 104 of the catheter 102, or even kinks against the distal portion of the catheter 102.

When a conventional expansion element is bent in relation to a longitudinal axis of a catheter, the input of coolant into the expansion element may become uneven, or even obstructed, thereby reducing the therapeutic value of a cryoablation procedure. For example, a bent expansion element may cause some portions of the expansion element to be closer to the input coolant than other portions of the expansion element, thereby causing the input coolant to significantly contact only some portions of the expansion element more than other portions of the expansion element, resulting in an undesired, uneven expansion of the expansion element.

The coiled distal end 214 of the coolant transfer tube 204 provides a structural rigidity, or stiffness, to the expansion element 108 to prevent bending of the expansion element 108 in relation to the distal portion 104 of the catheter 102. In at least some embodiments, the coiled distal end 214 may also be configured and arranged to maintain a position in the middle of the expansion element 108, thereby ensuring a spray of coolant that is directed to the desired one or more portions of the expansion element 108 during operation.

The stiffness of the coiled distal end 214 may also facilitate guidance of the catheter 102, during insertion, during operation, or during removal. As discussed above, the coiled distal end 214 is wrapped around a portion of the guide tube 202. The guide tube 202 is typically formed from a flexible material. When a stiffening member (e.g., a stylet or the like) is not inserted into the guide tube 202 (e.g., when the expansion element 108 is at or near the target location), application of one or more lateral forces collectively large enough to bend the expansion tube 108 in relation to the distal portion 104 of the catheter 102 may cause the expansion element 108 to kink against the distal portion 104 of the catheter 102. Kinking of the expansion element 108 may obstruct the flow of coolant from the expansion element 108 to the coolant outtake region 106, thereby causing a detrimental spike in pressure in the expansion element 108, eventually resulting in the rupturing of the expansion element 108.

The coiled distal end 214 of the coolant transfer tube 204 provides a structural rigidity, or stiffness, to the expansion element 108 to prevent kinking of the expansion element 108 against the distal portion 104 of the catheter 102. In some embodiments, the stiffness of the coiled distal end 214 is constant. In other embodiments, the stiffness of the coiled distal end 214 is variable. In at least some embodiments, the stiffness of the coiled distal end 214 is configured and arranged to smooth the transition from the distal portion 104 of the catheter 102 to a distal end of the expansion element 108.

In at least some embodiments, the stiffness is determined, at least in part, by the type of material used to form the coiled distal end 214. In at least some embodiments, a plurality of materials are used to form the coiled distal end 214. In at least some embodiment, the materials vary by stiffness over a longitudinal length of the coiled distal end 214. In at least some embodiments, the stiffness is determined, at least in part, by the amount of material used to form the coiled distal end 214. In at least some embodiments, thicker layers (or additional layers) of material may be used along one or more regions of the longitudinal length of the coiled distal end 214 to provide additional stiffness at desired locations.

Many different rigid, or semi-rigid, materials may be used to form the coiled distal end 214 including, for example, one or more metals (e.g., stainless steel), plastics, polyimides, or the like or combinations thereof. In at least some embodiments, at least a portion of the coolant transfer tube 204 disposed in the catheter 102 is formed from the same material(s) as the coiled distal end 214, thereby providing additional stiffness along at least a portion of the catheter 102.

In at least some embodiments, the stiffness is determined, at least in part, by the pitch of the coils. As the pitch of the coils increases, the coils are more separated from one another. Consequently, the coiled distal end 214 becomes more flexible as the pitch increases. In some embodiments, the coiled distal end 214 has a constant pitch. In other embodiments, the coiled distal end 214 is variably pitched. For example, in at least some embodiments, one end of the coiled distal end 214 has a first pitch and the opposite end of the coiled distal end 214 has a second pitch that is greater than the first pitch.

As another example, in at least some embodiments a middle section of the coiled distal end 214 is coiled at a first pitch 304, and proximal end and a distal end of the coiled distal end 214 are each coiled at a second pitch 306 that is greater than the first pitch 304. In at least some embodiments, the first pitch 304 is equal to a diameter of the coolant transfer tube 204. In at least some embodiments, the spray apertures 302 are positioned on the section of the coiled distal end 214 having the first pitch 304. In at least some embodiments, the spray apertures 302 are positioned along at least a portion of at least two differently-pitched sections of the coiled distal end 214. In at least some other embodiments, the spray apertures 302 are positioned along the entire length of the coiled distal end 214.

Typically, the catheter 102 is inserted in patient vasculature and guided to a target location, such as the ostia of the pulmonary veins in the left atrium of the heart of the patient. Coolant from the coolant source 106 may be released into the catheter 102. In at least some embodiments, the coolant source 106 includes a pressurized container or pump. In at least some embodiments, the lower pressure in the expansion element 108 draws the coolant along the coolant transfer tube 104 and into the expansion element 108. In at least some embodiments, the fluid-drawing source 114 may be used to draw the coolant from the expansion element 108 back out of the catheter 102 along the coolant outtake region 206. The rate of flow of the coolant within the catheter 102 may be adjusted to a rate appropriate to the specific type of operation.

In at least some embodiments, the expansion element 108 is inflated to a pressure that is no more than 2 atm (about 2×10⁵ Pa). In at least some embodiments, the expansion element 108 is inflated to a pressure that is no more than 3 atm (about 3×10⁵ Pa). In at least some embodiments, the expansion element 108 is inflated to a pressure that is no more than 4 atm (about 4×10⁵ Pa). In at least some embodiments, the expansion element 108 is inflated to a pressure that is no more than 5 atm (about 5×10⁵ Pa). In at least some embodiments, the expansion element 108 is inflated to a pressure that is no more than 6 atm (about 6×10⁵ Pa). In at least some embodiments, the expansion element 108 is inflated to a pressure that is no more than 7 atm (about 7×10⁵ Pa). In at least some embodiments, the expansion element 108 is inflated to a pressure that is no more than 8 atm (about 8×10⁵ Pa). In at least some embodiments, the expansion element 108 is inflated to a pressure that is no more than 9 atm (about 9×10⁵ Pa).

In at least some embodiments, the temperature of the expansion element 108 is reduced to a temperature that is no greater than −20° C. In at least some embodiments, the temperature of the expansion element 108 is reduced to a temperature that is no greater than −40° C. In at least some embodiments, the temperature of the expansion element 108 is reduced to a temperature that is no greater than −60° C. In at least some embodiments, the temperature of the expansion element 108 is reduced to a temperature that is no greater than −80° C. In at least some embodiments, the temperature of the expansion element 108 is reduced to a temperature that is no greater than −100° C. In at least some embodiments, the temperature of the expansion element 108 is reduced to a temperature that is no greater than −120° C. In at least some embodiments, the temperature of the expansion element 108 is reduced to a temperature that is no greater than −140° C. In at least some embodiments, the temperature of the expansion element 108 is reduced to a temperature that is no greater than −160° C. In at least some embodiments, the temperature of the expansion element 108 is reduced to a temperature that is no greater than −180° C. In at least some embodiments, the temperature of the expansion element 108 is reduced to a temperature that is no greater than −200° C.

In at least some embodiments, the temperature within the expansion element is reduced from an insertion temperature to an operational temperature over a time period that is no greater than one minute. In at least some embodiments, the temperature within the expansion element is reduced from an insertion temperature to an operational temperature over a time period that is no greater than two minutes. In at least some embodiments, the temperature within the expansion element is reduced from an insertion temperature to an operational temperature over a time period that is no greater than three minutes. In at least some embodiments, the temperature within the expansion element is reduced from an insertion temperature to an operational temperature over a time period that is no greater than four minutes. In at least some embodiments, the temperature within the expansion element is reduced from an insertion temperature to an operational temperature over a time period that is no greater than five minutes.

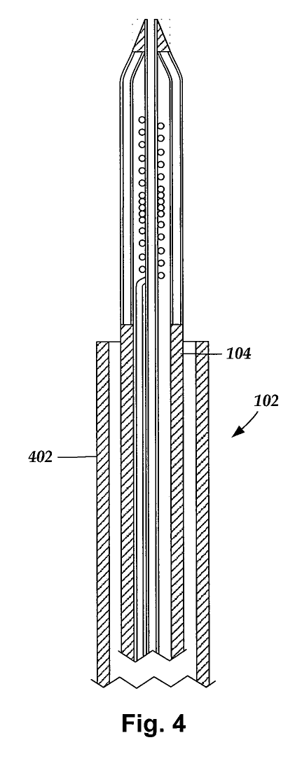

In at least some embodiments, a sheath may be used to facilitate guidance of the catheter through patient vasculature during insertion of the catheter. FIG. 4 is a schematic longitudinal cross-sectional view of one embodiment of the distal portion 104 of the catheter 102 disposed in a sheath 402. In at least some embodiments, the sheath 402 is steerable. Once the catheter 102 is positioned at a target location, such as the ostia of the pulmonary veins in the left atrium of the heart of the patient, the sheath 402 can be removed. When a pulmonary vein is the target location, the expansion element 108 can be expanded to occlude the lumen of the pulmonary vein. In at least some embodiments, the expansion element 108 may be expanded with a non-cryoablating fluid so as to not damage patient tissue. A contrast agent may be injected into the pulmonary vein and the region can be imaged to assess the location of the expansion element 108 and the ability of the expansion element 108 to occlude the vessel (thereby determining the potential efficacy of a cryoablation procedure by determining how well the expansion element 108 contacts the walls of the vessel). Coolant can be input to the catheter 102 to begin the tissue ablation.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. A cryoablation catheter assembly comprising:
a catheter having a distal portion, a proximal portion, and a longitudinal length, the catheter configured and arranged for insertion into patient vasculature, the catheter comprising
a guide tube extending along the catheter, the guide tube extending beyond the distal portion of the catheter;
a coolant transfer tube extending along the catheter, the coolant transfer tube comprising a coiled distal end extending beyond the distal portion of the catheter, the coolant transfer tube defining a lumen configured and arranged to receive and transfer coolant from a coolant source, the coiled distal end defining a plurality of spaced-apart spray apertures, wherein the coiled distal end is wrapped around a portion of the guide tube; and
at least one coolant outtake region extending along the catheter; and
an expansion element coupled to the distal portion of the catheter, the expansion element in fluid communication with the at least one coolant outtake region, wherein at least a portion of the coiled distal end of the coolant transfer tube extends into the expansion element such that the expansion element is in fluid communication with the plurality of spray apertures.

2. The cryoablation catheter assembly of claim 1, wherein the coiled distal end of the coolant transfer tube provides stiffness to the expansion element.

3. The cryoablation catheter assembly of claim 1, wherein a distal end of the expansion element is coupled to a distal end of at least one of the guide tube or the coiled distal end of the coolant transfer tube.

4. The cryoablation catheter assembly of claim 1, wherein the coiled distal end of the coolant transfer tube is centered along a longitudinal axis of the expansion element.

5. The cryoablation catheter assembly of claim 1, wherein the expansion element comprises an inner member and an outer member disposed over the inner member.

6. The cryoablation catheter assembly of claim 1, wherein the plurality of spray apertures are configured and arranged such that, when coolant is passed through the plurality of spray apertures from the coolant transfer tube, the coolant either sprays substantially evenly against the expansion element, or sprays substantially evenly against one or more localized portions of the expansion element.

7. The cryoablation catheter assembly of claim 1, wherein the expansion element is configured and arranged to transition from a deflated configuration to an inflated configuration when coolant is output from the plurality of spray apertures.

8. The cryoablation catheter assembly of claim 7, wherein the coiled distal end of the coolant transfer tube extends to a distal end of the expansion element when the expansion element is in the deflated configuration.

9. The cryoablation catheter assembly of claim 7, wherein the coiled distal end of the coolant transfer tube extends to a distal end of the expansion element when the expansion element is in the inflated configuration.

10. The cryoablation catheter assembly of claim 1, wherein a distal end of the guide tube extends beyond the coiled distal end of the coolant transfer tube.

11. The cryoablation catheter assembly of claim 1, wherein the coiled distal end of the coolant transfer tube comprises at least one section with a first pitch and at least one section with a second pitch that is greater than the first pitch.

12. The cryoablation catheter assembly of claim 1, wherein the coiled distal end of the coolant transfer tube comprises at least one section formed from a first material and at least one section formed from a second material that has a greater stiffness than the first material.

13. The cryoablation catheter assembly of claim 1, wherein the coiled distal end of the coolant transfer tube extends into the distal portion of the catheter.

14. The cryoablation catheter assembly of claim 1, wherein the coiled distal end of the coolant transfer tube has a stiffness that is sufficient to prevent kinking of the expansion element with the distal portion of the catheter when the expansion element is subjected to lateral forces large enough to bend the expansion element in relation to the distal portion of the catheter.

15. A cryoablation system comprising:
a catheter having a distal portion, a proximal portion, and a longitudinal length, the catheter configured and arranged for insertion into patient vasculature, the catheter comprising
a guide tube extending along the catheter, the guide tube extending beyond the distal portion of the catheter,
a coolant transfer tube extending along the catheter, the coolant transfer tube comprising a coiled distal end extending beyond the distal portion of the catheter, the coolant transfer tube defining a lumen configured and arranged to receive and transfer coolant from a coolant source, the coiled distal end defining a plurality of spaced-apart spray apertures, wherein the coiled distal end is wrapped around a portion of the guide tube, and
at least one coolant outtake region extending along the catheter;
an expansion element coupled to the distal portion of the catheter, the expansion element in fluid communication with the at least one coolant outtake region, wherein at least a portion of the coiled distal end of the coolant transfer tube extends into the expansion element such that the expansion element is in fluid communication with the plurality of spray apertures;
a coolant source coupled to the coolant transfer tube;
a fluid-drawing source coupled to the at least one coolant outtake region; and
a control module coupled to the catheter, the coolant source, and the fluid-drawing source, wherein the control module comprises a coolant flow controller configured and arranged for controlling the flow of coolant along the coolant transfer tube and the at least one coolant outtake region.

16. The cryoablation system of claim 15, wherein the control module further comprises a pressure sensor for monitoring pressure within the catheter.

17. The cryoablation system of claim 15, wherein the control module further comprises a temperature sensor for monitoring temperature within the catheter.

18. The cryoablation system of claim 15, wherein the coolant source comprises a coolant, the coolant being a liquefied gas under pressure.

19. A method for cryoablating patient tissue, the method comprising:
inserting a catheter in patient vasculature, the catheter having a distal portion, a proximal portion, and a longitudinal length, the catheter comprising a guide tube extending along the catheter, a coolant transfer tube extending along the catheter, and at least one coolant outtake region, wherein the guide tube and the coolant transfer tube extend beyond the distal portion of the catheter, and wherein the coolant transfer tube comprises a coiled distal end that wraps around a portion of the guide tube;
guiding the catheter in proximity to patient tissue to be ablated;
drawing coolant from a coolant source such that coolant flows along the coolant transfer tube and is sprayed from spaced-apart spray apertures defined in the coiled distal end of the coolant transfer tube against an expansion element that is disposed at the distal portion of the catheter and that surrounds the coiled distal portion of the coolant transfer tube, thereby expanding the expansion element and reducing the temperature of the expansion element to a temperature sufficiently low enough to ablate patient tissue upon contact; and
contacting patient tissue with the expanded expansion element for a time period adequate to ablate tissue contacting the expansion element.

20. The method of claim 19, wherein guiding the catheter in proximity to patient tissue to be ablated comprises disposing the catheter into a steerable sheath.
